(19) United States
(12) Patent Application Publication
(10) Pub. No.: US 2005/0069949 A1
(43) Pub. Date: Mar. 31, 2005

(54) Microfabricated Fluidic Structures

(75) Inventors: JAMES N. HUMENIK, LAGRANGEVILLE, NY (US); GOVINDARAJAN NATARAJAN, POUGHKEEPSIE, NY (US); SANDRA WHALEY, HOUSTON, TX (US); ALBERT YOUNG, FISHKILL, NY (US)

Correspondence Address: INTERNATIONAL BUSINESS MACHINES CORPORATION;DEPT. 18G, BLDG. 300-482, 2070 ROUTE 52, HOPEWELL JUNCTION, NY, 12533, US

(73) Assignee: INTERNATIONAL BUSINESS MACHINES CORPORATION, ARMONK, NY (US)

(21) Appl. No.: 10/605,430

(22) Filed: Sep. 30, 2003

(51) Int. Cl. G01N 33/53; G01N 33/543

(52) U.S. Cl. 435/7.1; 436/518

(57) ABSTRACT

A plate for use in mixing and testing materials in the pharmaceutical industry is formed by a method in which an array of sample cells contain a U-shaped structure having two vertical apertures connected by a horizontal passage in a bottom sheet; reagents are drawn in to the vertical passages by capillary action or other forces and react in the horizontal passage. An optional version of the invention includes a relatively large reservoir for containing rinsing fluids.

### CROSS REFERENCE To RELATED APPLICATIONS

This application relates to the invention described in Attorney Docket Number FIS920020186US1, incorporated herein by reference in its entirety.

### BACKGROUND OF INVENTION

Technical Field

The field of the invention is that of simultaneously testing many compounds for biological/chemical interactions. In particular, the current invention is a device/structure and a method to test drug interactions.

In order to improve the efficiency of drug discovery, leading pharmaceutical companies have implemented high; throughput screening (HTS) techniques for the evaluation of potential drug candidates. In high throughput screening, a reagent set A (for example, a biological target with appropriate assay reagents) is tested for reactivity with chemicals B1-Bn (for example, compounds taken from a molecular library), where n can be a large number, on the order of millions. High-throughput screening can enable the testing of large numbers of compounds rapidly and in parallel. Current efforts are standardized around the use of plastic consumables known as microtiter plates, or microplates. A set of substances B1-Bn can be arrayed in these microplates, and then reagent set A, which could include chemicals that test for the interaction with a specific biological target, can be mixed with each of the Bn. Detector instrumentation, for example, optical microplate readers, can then be used to detect interactions.

The pharmaceutical industry currently has a need for improvements in high-throughput-screening technology to improve drug-discovery efficiency and to keep costs down. Reagents and compounds used in drug discovery are often scarce and expensive, which has prompted the development of miniaturized assays with smaller assay volumes. Microtiter plates are commercially available in a variety of standard well formats (e.g. 96;- 384, and 1536 wells per plate), with well dimensions typically on the order of a few to several millimeters. Assays performed in these plates typically use in excess of ten microliters of reagent per test point. These types of reactions could theoretically be performed with sub-microliter volumes of reagents, but to date such low-volume assays have not achieved widespread adoption. One significant factor inhibiting the adoption of low-volume assays is the lack of methods for reliable high-performance fluid delivery.

Recently, Autonomous Microfluidic Capillary System David Juncker, Heinz Schmid, Ute Drechsler, Heiko Wolf, Marc Wolf, Bruno Michel, Nico de Rooij, and Emmanuel Delamarche, Anal. Chem.; 2002; 74(24) pp 6139-6144; has described a specific design concept to regulate the flow of multiple reagents in a capillary-driven microstructure. In this concept, the flow of a reagent is initiated by its delivery to a service port and then terminates when the fluid has drained to the point where the trailing meniscus has reached an element known as a capillary retention valve. Flow rates during this phase can be controlled by engineering the geometry and surface characteristics of the microstructure.

The art has been able to provide some control over the position of the liquid in a microstructure, but the user is required to deposit the correct amount of fluid into the service port, with a high degree of accuracy. One of the difficulties in moving to smaller and smaller amounts of liquid is the ability to meter out precise quantities of liquid for delivery into the service port using conventional means. A method is needed whereby the microstructure can actually improve the delivery of fluid instead of merely acting as a receiver.

### SUMMARY OF INVENTION

The invention relates to a device with micro wells and micro channels and a method for formation thereof.

In one aspect of the invention, the open wells and channels are formed by individual layer personalization.

In another aspect of the invention, the array comprises U-shaped channels with vertical branches having different diameters.

In another aspect of the invention, fluid delivery in the channels is controlled with engineered geometries in the channels.

In another aspect of the invention, fluid delivery is controlled by parameters of various surfaces and/or surface features like roughness.

In another aspect of the invention, self-metering of fluid volume is achieved by use of differential capillary forces.

Another feature of the invention is the use of a sacrificial material that escapes from the ceramic structure during the sintering process.

Another aspect of the invention is the control of the channel volume during sintering process.

Another aspect of the invention is a set of methods for reacting reagents in such a device.

Another aspect of the invention is a set of methods for delivering reagents and materials to surfaces using such a device.

Another aspect of the invention is a method of metering the delivery of reagents into such a device.

### BRIEF DESCRIPTION OF DRAWINGS

FIG. 1 shows a general view of an embodiment of the invention together with a holder for the embodiment.

FIG. 2 shows a top view of an array.

FIG. 3 shows a detail of a sub-array.

FIG. 4A shows a top view of an individual module of an array.

FIG. 4B shows a cross section of the module of FIG. 4A.

FIGS. 5A-5G show steps in the assembly of an embodiment of the invention.

FIG. 6 shows a step in fluid delivery.

FIGS. 7A-7D show transfer control implemented by surface parameters.

FIGS. 8A-8G illustrate steps in a sequence of operations that transfer a defined quantity of reagent.

FIG. 9A shows a top view of an embodiment of the invention.

FIG. 9B shows a cross section of the module of FIG. 4A.

FIG. 10 shows a detail of an assembly of an embodiment of the invention.

FIG. 11 shows an arrangement adapted for optical inspection of the array.

FIG. 12 shows an alternative array adapted for optical inspection and mechanical manipulation.

FIG. 13 shows an exploded view of a detail of a holder according to the invention.

### DETAILED DESCRIPTION

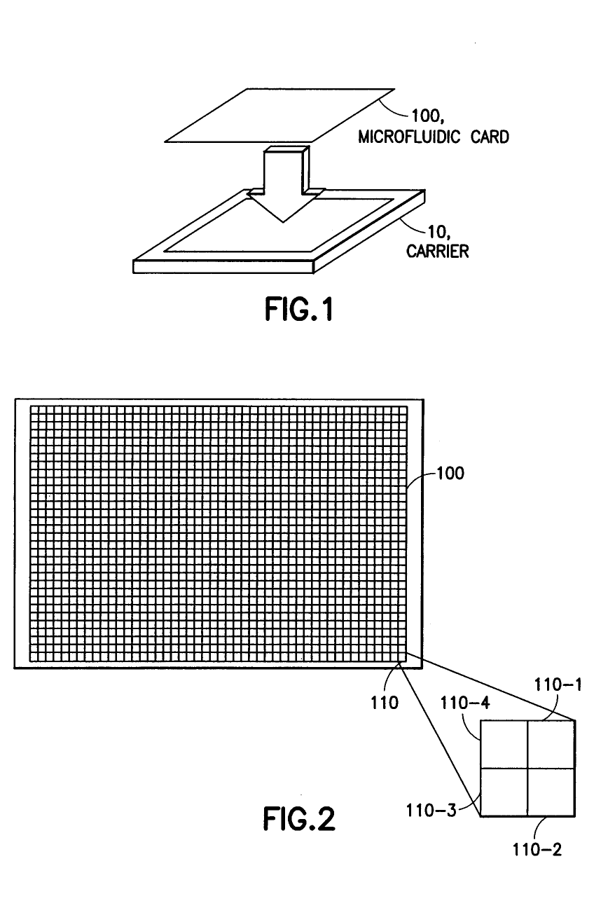

FIG. 1 shows a perspective view of an embodiment of the invention together with a holder/carrier for it. Card 100, according to the invention, is a relatively thin plate containing an array of fluid containers that contain a set of samples to be tested. The overall dimensions of card 100 are in compliance with an industry standard, as are the location of the individual modules within the array.

Card 100 fits into holder 10, which positions it, has available vacuum and pressure for fluid control and adapts to a robotic material handling apparatus.

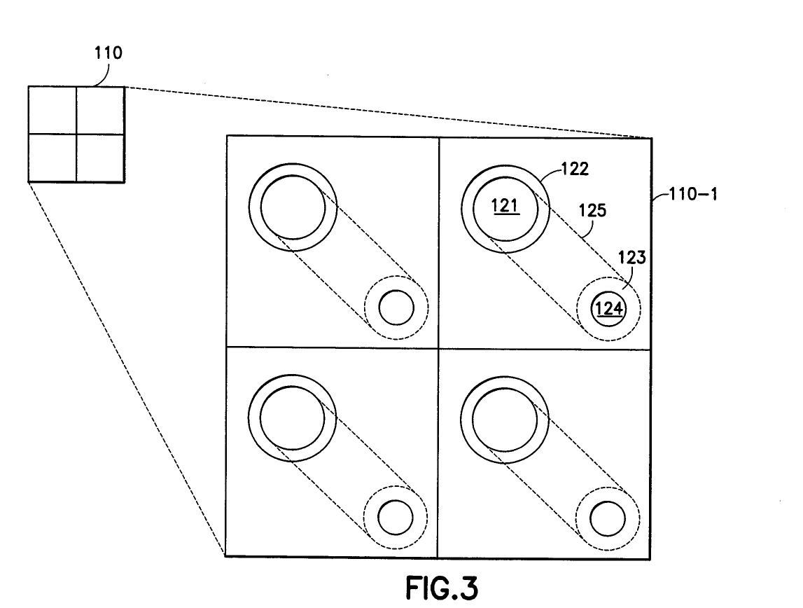

FIG. 2 shows a top view of an array 100 according to the invention. The industry has defined specifications for standard arrays, though non-standard arrays may be used if preferred. In this case, the array is a set of 48×32 sub-modules, each of the sub-modules containing a 2×2 sub-array of unit modules. On the lower right of the Figure, a sub-module 110 contains four unit modules 110-1-110-4 that are illustrated in the following Figures.

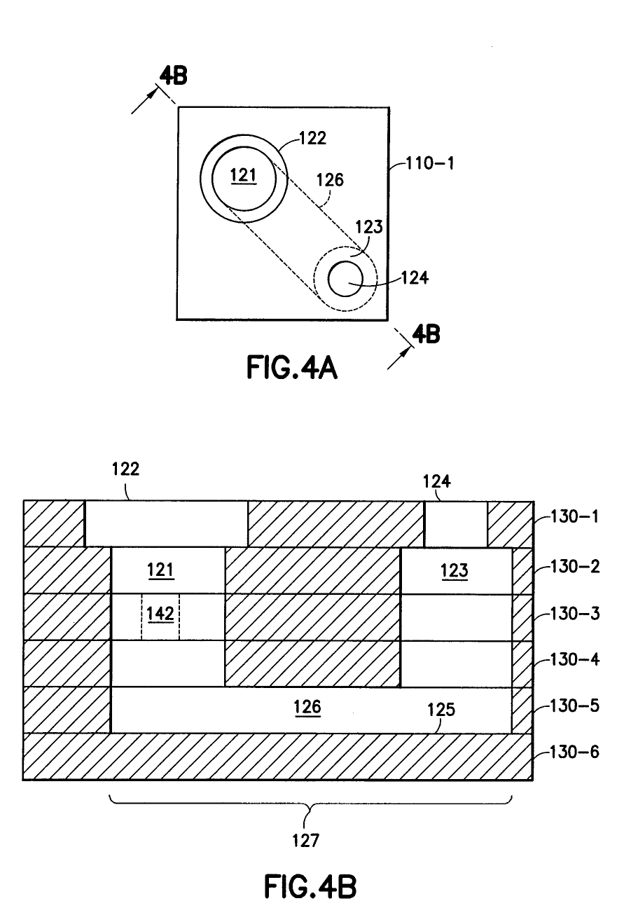

FIG. 3 shows a detail of a sub-module 110, containing four unit modules 110-1-110-4. Each unit module contains a U-shaped channel with one larger input branch and one smaller output branch. For example, the input branch has a top diameter denoted by a circle 122 and a vertical passage 121. The output branch has a top diameter 124 and a vertical passage 123. The diameter of passage 123 is shown as being the same diameter for convenience in the drawing, but may be less than the corresponding diameter of passage 121. The sizes of the branches and surface materials of the branches are chosen as described below, to control fluid motion and position.

One novel example of the use of U-shaped geometries to help achieve reproducible microfluidic device performance stems from their ability to help prevent the introduction of undesired bubbles into the active device regions of a microfluidic structure.

The invention takes advantage of microfluidic separation by gravity, relying on the fact that bubbles that are introduced at the input of a device will float up to the top. So a geometry that allows bubbles to float to the top and where the bubble-free fluid can then be directed downward to the active device areas assists in excluding bubbles from active regions. The use of U-shaped structures is one method to prevent bubble incorporation into the microchannel. Other methods such as size-exclusion filters can be implemented in conjunction with this approach to assist in the removal of bubbles from specified areas.

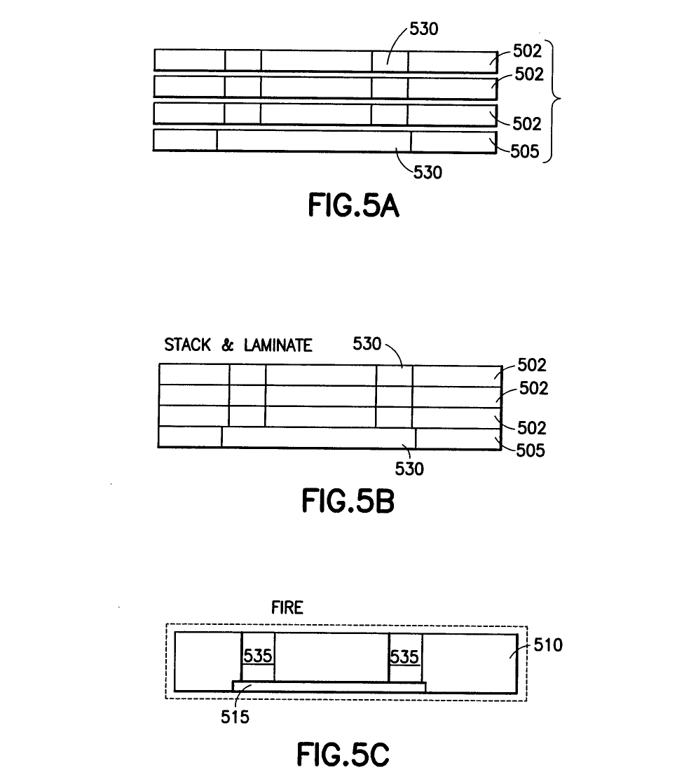

FIG. 4A shows a top view of a single sub-module of the array in FIG. 2. FIG. 4B shows a cross section of the structure of FIG. 4A, formed using 6 green sheets and 1 horizontal channel connecting two vertical wells for simplicity in illustration. It should be noted that both vertical wells and horizontal channels can be formed in a single layer or by combination of multiple layers in a suitable material, like ceramic, organic, glass, metal, or composite. The structure shown in FIG. 4 has been assembled from individual sheets by lamination. The assembly process is the same for ceramic structures with arrays of thousands of unit modules, with thousands of horizontal channels selectively connected to link vertical holes. The ceramic material may include alumina, glass ceramic, aluminum nitride, borosilicate glass and glass. The diameter of vertical wells 121, 123 can be 20 microns or more, the channel width 126 can be 20 microns or more and the length can be a minimum of two diameters/40 microns. The foregoing dimensions are illustrative and may decrease as technology improves. The shape of a well exposing a substance may be circular, rectangular, smooth or rough. The total thickness of the plate 100 may be any desired amount, but preferably is under 1 mm. The thickness of an individual greensheet depends on the application, but preferably is about 150 microns.

The lamination process involves heat, pressure and time. The preferred lamination pressure is under 800 psi, the temperature is under 90 deg C. and for a time of less than 5 minutes. The sintering process involves the material of choice and the binder system used to form the greensheets.

The sintering process could include temperatures less than 2000C, and can be isostatic, free, and/or conformal. The ambient includes air, nitrogen, hydrogen, steam, carbon dioxide, and any combinations thereof.

The diameter of channels used in fabrication will depend on the particular application and technical variables such as the viscosity of the substance passing through, the surface tension/activity of the surface and fluid, desired flow force, capillary or forced flow, desired quantity and rate of flow, etc.

According to one example of the invention, the greensheets are formed from a substance such as alumina, glass, ceramic and glass and ceramic, referred to as ceramic greensheets. The technique for forming vertical apertures and horizontal channels is material removal by mechanical techniques such as punching the material out, laser drilling, e-beam drilling, sandblasting and high pressure liquid jets. Some applications may employ channels formed by non-material-removal techniques such as embossing, pressing, forming, and casting.

FIG. 4B shows a portion of a simplified completed structure according to the invention, formed of six layers and having a single horizontal channel 126 formed in a sheet 130-5 and connecting two vertical apertures 121 and 123 formed in sheets 130-2, 130-3 and 130-4. The sheets 130-i were initially separate ceramic greensheets that have been laminated and sintered in a conventional process to form ceramic plate 100. At the top, different sized apertures described below are used for input of fluid reagents and for input of another reagent that combines to form the sample or for application of the test compound for the test of the compound.

In one embodiment of the invention, the layer that contains the bottom surface of the horizontal channel 126 has the bottom surface of the channel adapted for holding sample material, e.g. reaction products. The surface may have a minimum roughness (of less than 1 micron, say) and/or be shaped with a depression to contain the material during handling. In addition, the layer should be adapted for high speed scanning, e.g. be thin enough to fit in conventional scanners, have the cells placed close enough together to minimize time spent traveling from one to another, etc. FIG. 4B shows a version in which the top surface 125 upon which reaction products will deposit (and that forms the bottom of the U-shaped structure) is in a solid bottom layer 130-6 and the aperture is formed in a lower layer 130-5 that rests on the bottom layer. An alternative in which aperture 126 is formed as a groove in the bottom layer may also be used.

Preferably, the layer containing the top surface 125 upon which reaction products will deposit (and that forms the bottom of the U-shaped structure) is removable; i.e. it adheres to the upper layers well enough to keep the fluids from leaking, but can easily be separated from the upper layers. The method of attachment may be any known in the art, e.g. heat, tape, a pressure-sensitive sealant, or silk-screening a sealing material.

In operation, a reagent is inserted (using a pipette for example) in aperture 122, then is attracted by the increased capillary force caused by the decrease in diameter down to passage 121. The reagent is drawn in for a set time after which the dispensing pipette is withdrawn.

When the reagent reaches the bottom of passage 121, it travels horizontally until it reaches passage 123, where it rises up to a level that may be influenced by various means described below.

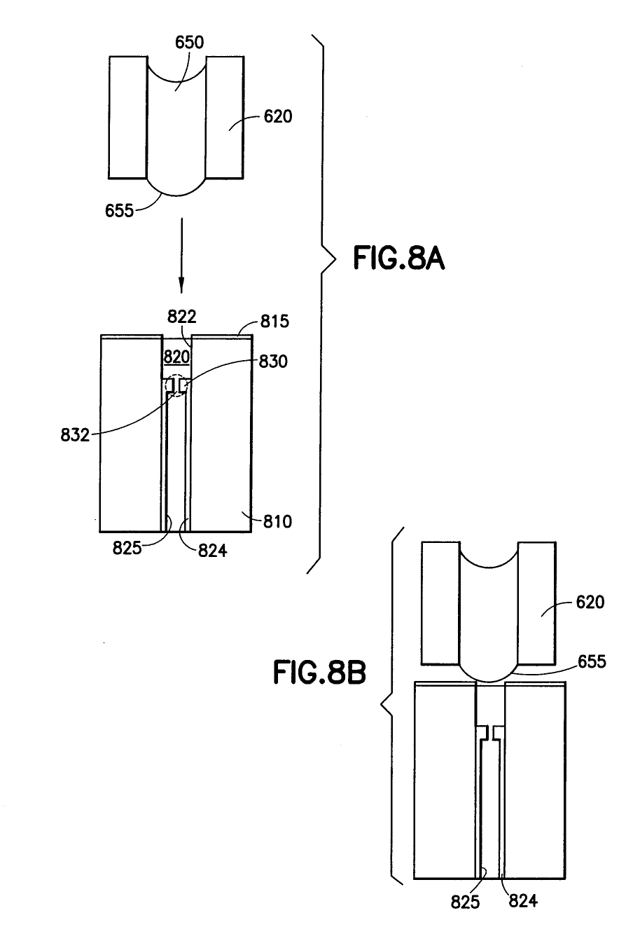

Referring to FIG. 8, the fluid can be effectively self-metered from an external fluid reservoir (as one example, we can use a conventional pipette tip), by designing a system whereby the structure works in conjunction with the fluid reservoir. This requires some limited knowledge of the external fluid reservoir's geometry, dimensions, and surface-wettability characteristics. The microstructure provides capillary pressure to draw in fluid by a combination of diameter and surface properties such that the capillary force pulling fluid into the reservoir is greater than the force keeping it in the pipette.

One embodiment provides a flow-resistance element to control the rate of fluid extraction. In typical use, the external fluid reservoir would be filled with an amount of liquid in excess to that amount actually required. By bringing the fluid in the pipette tip into contact with the microfluidic device, flow is initiated. The flow rate is regulated by the flow-restriction element, so the desired volume can be achieved by controlling the amount of time that the pipette tip interacts with the microfluidic device. The pipette tip can then be removed from proximity with the microfluidic card to terminate the metering operation. The fluid will then flow until it has self-positioned itself with its trailing meniscus at the position known as the capillary retention valve (CRV) denoted by numeral 820 where a restricted diameter operates to resist further flows.

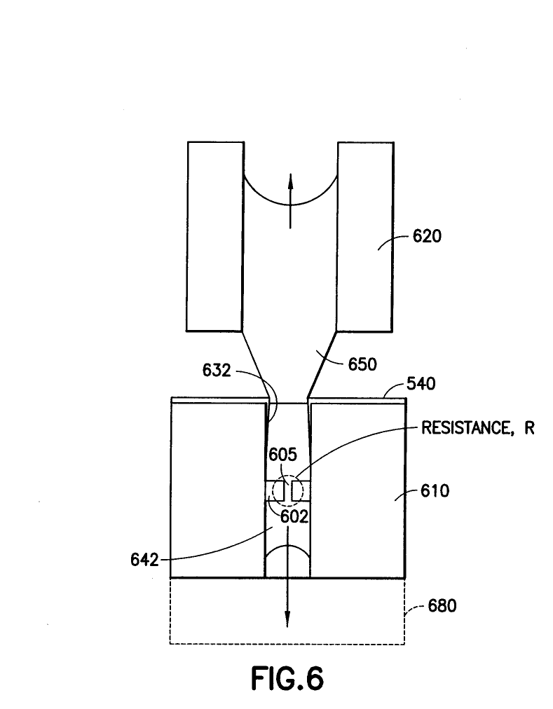

FIG. 6 shows the basic operation, in which a pipette 620 is brought into proximity to a unit cell in an array 610. When the projecting portion of the fluid touches the aperture, capillary force initiates flow from the pipette into the channel. The attraction may be aided by making the inner surface of the receiving channel one that is wetted by the fluid and the top surface 540 one that is not wetted. (This also reduces spillage.) The fluid passes into the channel and through the restriction aperture 605 in restriction member 602, which is sized to reduce the fluid flow so that a timed flow will be more accurate. After the specified time, which will depend on the fluid viscosity, the dimensions and surface properties of the pipette and receiving channels, including the restriction aperture and the desired volume to be transferred, the pipette is removed. The fluid settles with its upper (trailing) meniscus at the restriction aperture.

FIG. 8A shows the initial approach of pipette 620 carrying fluid 650 with projecting fluid 655 to the cell 810, which has aperture 820 with upper interior surface 822, top surface 815 and restriction aperture 832 in CRV 830. Below the restriction aperture, the inner surface 825 of liner 824 has a different (and greater) attraction for the fluid than the upper surface 822.

FIG. 8B shows the projection of the projecting fluid 655 just touching the top of aperture 820.

In FIG. 8C, the fluid is in the initial stage of transfer, with a lower meniscus 662 approaching the restriction aperture 832.

FIG. 8D shows the stage after the lower meniscus has passed through the restriction aperture and is passing down the lower portion of aperture 820 (the storage reservoir) at a rate determined largely by restriction aperture 832.

FIG. 8E shows the same structure after the pipette has been withdrawn, with drop 655 at the bottom of the pipette having been separated from the top surface 655.

FIG. 8F shows the structure shortly after, when more of the fluid has passed into the lower storage reservoir, with lower meniscus 664 having passed to a lower depth and an upper meniscus 672 having formed.

Lastly, FIG. 8G shows the structure in its final state, when the upper meniscus 672 has been pinned at the level of the restriction aperture.

The operation has been shown with a single vertical aperture for simplicity, but the U-shaped structure of FIG. 4 or more complex structures may be used.

One area where these techniques are applicable is in the area of reagent storage. Useful reagent storage (whether for minutes or months) at small volumes is complicated by the difficulty of controlling the positioning of fluid within the storage container. When there is poor control over initial positioning of stored reagents, subsequent reactions of these reagents with additional reactants are not well controlled. According to the invention, microfluidic structures with integrated capillary-retention valves may be used for reagent storage. Using this method, reagents can be applied to the inlet port of a microstructure with relatively low precision, but can then be precisely driven by capillary action to move fluid to a predetermined position within the microstructure.

Referring again to FIG. 6, the lower portion of the vertical channel may be used to store a reagent, with the trailing meniscus pinned to aperture 605 holding it in place. The vertical channel of FIG. 6 can be part of a U-shaped structure as in FIG. 4 or of a more complex structure. The accurate positioning of the fluid enables one to calculate precisely the dynamics of a reaction so that it is reproducible and as designed. Such reagents stored in microstructures can also be held or frozen in situ for use at much later times.

The rinsing of fluids is an important step in many biochemical protocols. However, achieving reproducible rinsing at low liquid volumes is difficult-commercially, an inherently large footprint per test is currently required to achieve good results. The ability to perform multiple fluid rinses in a small footprint would be advantageous and a method to do so within a microstructure has been demonstrated in the literature. However, in that instance, a separate secondary structure is needed in order to enable fluid extraction (which drives the rinse process by a capillary-flow mechanism) from the primary fluid-processing microstructure. This requirement for a secondary component adds undesired complexity (e.g. alignment requirements) to practical implementations. According to the invention, a fully-integrated structure is able to perform rinsing and to enable multistep assays by using multilayer structures to significantly increase the volume of the attached capillary-driven flow-promotion zone (esp. in the third dimension). Illustratively, an optional feature of FIG. 6 is an additional set of greensheets denoted generally by dotted line 680 that adds a longer and deeper reservoir at the bottom of FIG. 6.

This method allows for a small overall footprint, enables low-volume assays that are heterogeneous in nature, and helps to prevent spillover of unwanted reagent in the event that the microfluidic structure is composed of multiple parts and needs to be separated.

Similar microfluidic methods and structures can be used to precisely deliver biological cells and other non-fluid entities (such as beads or nanoparticles) carried in a non-homogenous fluid to a substrate. The substrate can, for example, be a wall of an assembled structure which can then be disassembled to allow substrate-specific processing. Also, reagents can be delivered to any such entities (e.g. cells, beads, nanoparticles, etc) that have been attached to a surface of the microchannel in an earlier step. As one example, culture media with biological cells can be delivered to a microstructure and positioned through the use of a capillary-retention valve. The biological cells can then settle to the bottom surface 125 of the microstructure (channel 126) in a predictable manner, where they are then to able attach themselves in a process similar to that found in conventional cell culture. Subsequent rinse and reagent application steps can then be used to perform valuable cell-based assays.

Conventional methods for low-volume reagent handling are generally very wasteful of reagents. This becomes especially problematic when a reagent is expensive and/or in short supply. Structures according to the invention use a microstructure with a height that is typically a reduced multiple of the diffusion constant (which must be at least roughly known) to minimize reagent that cannot interact with the surface. Additionally it provides for a designed flow using the techniques described above, such that in approximately the amount of time it takes for reagents to be depleted near the surface, a fresh supply of reagent can be introduced. This can be either continuous or quantized flow (periods of flow separated by periods when flow is stopped), but the design is intended to allow the most efficient application of reagent in the shortest time. The invention also includes use of microfluidic structures to write lines and spots in which a projecting drop such as 655 in FIG. 8A is brought into contact with the paper or other medium.

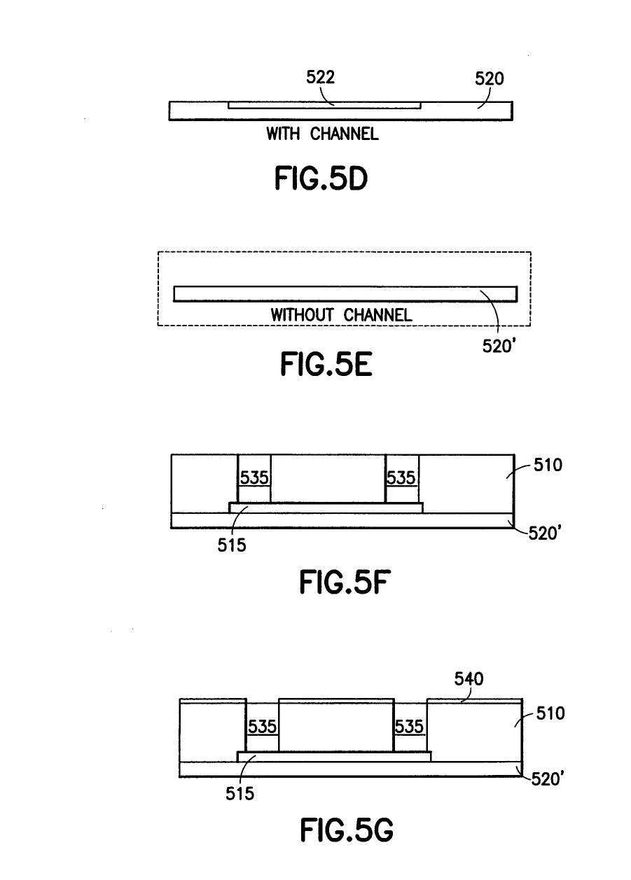

Referring now to FIG. 5, there is shown the sequence of assembling an embodiment of the invention, in which FIG. 5A shows three ceramic greensheets 502 stacked up, each greensheet containing a fugitive material 530 filling the site of a vertical aperture. At the bottom, sheet 505 contains a horizontal strip, also filled with material 530, that will become a horizontal channel connecting the two vertical apertures. FIG. 5B shows the assembled stack, ready for firing and FIG. 5C shows the assembly 510 after firing, with the U-shaped passage comprising the two vertical passages 535 and the horizontal passage 515.

In FIGS. 5D and 5E, two variants of a bottom plate are shown, with plate 520 in FIG. 5D having a channel 522 formed into its upper surface and plate 520′ in FIG. 5E without a channel.

FIG. 5F shows the combination of the assembly of FIG. 5C with the bottom plate 520′ of FIG. 5E.

FIG. 5G shows the assembly after an optional step of treating the top surface with a substance 540, illustratively to prevent a reagent from wetting the top surface and wasting reagent that will not pass into one of the apertures 535. Those skilled in the art will appreciate that other topologies are possible, for example that more than one vertical aperture may be formed, that a restriction aperture such as that shown in FIG. 6 may be included in one or both vertical apertures and that one or more vertical apertures may extend down below the horizontal aperture 515 for storage of rinsing fluid or excess reagent.

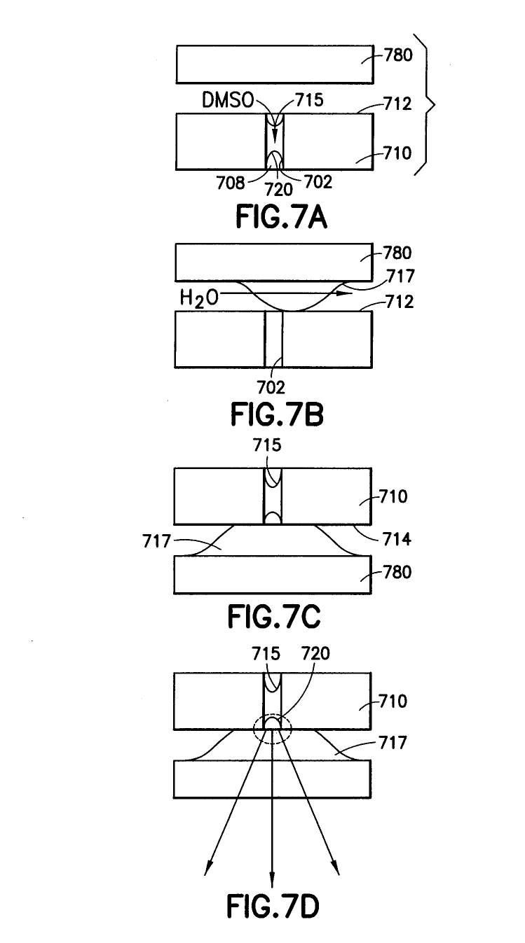

FIG. 7 shows a sequence illustrating the use of differential wettability. FIG. 7A shows a single aperture 708 in plate 710, having received a quantity of reagent dissolved in, for example, dimethylsulfoxide DMSO, a conventional solvent. Interior surface 702 of the aperture has been treated (or the material of block 710 has been chosen) to attract the DMSO through capillary force.

In contrast, as shown in FIG. 7B, top surface 712 of block 710 is not wetted by water and water-based reagents will not penetrate into the channel. FIG. 7C shows the administration of a water-based reagent from below, so that the fluid penetrates into the aperture from below. The volume of DMSO fluid has been chosen such that the lower meniscus 720 will be reached by the water-based reagent 717. As shown in FIG. 7D, the two fluids meet and react in an overlap zone denoted by the dashed line in FIG. 7D.

The parameters have been chosen such that the diffusion distances of the reagents permit the reactants to reach one another.

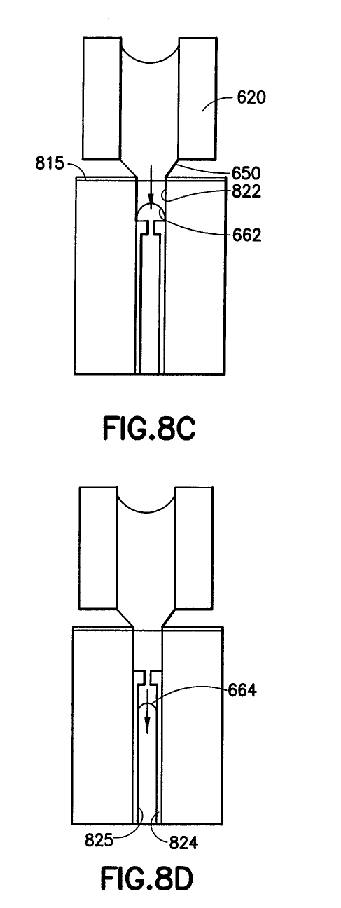

Referring now to FIG. 9A, there is shown a top view looking toward the x-y plane, of a holder according to the invention, in which a frame 150 holds the micro-plate. Frame 150 translates in the x and y directions as discussed below. On the left, box 135 represents a battery that supplies electrical power to actuators. Alternatively, box 135 could represent a storage unit for compressed gas for application to actuators and/or to the modules in the array to move fluids in or out.

Numeral 55 represents a ledge that holds the microplate. Numeral 52 denotes a large aperture that exposes the array of wells to operations implemented from below. Tubes 42 and 44 represent gas and vacuum lines. At the corners, boxes 120 represent position sensors for the measurement of alignment of the microplate.

FIG. 9B shows a cross section of the holder of FIG. 9A, in which plate 50 is shown as displaced from ledge 55. Lifting pins 45 represent a feature for raising the plate so that robotic material handlers can grip it. Lower frame 110 contains actuators described below for moving frame 150 in the x-y plane.

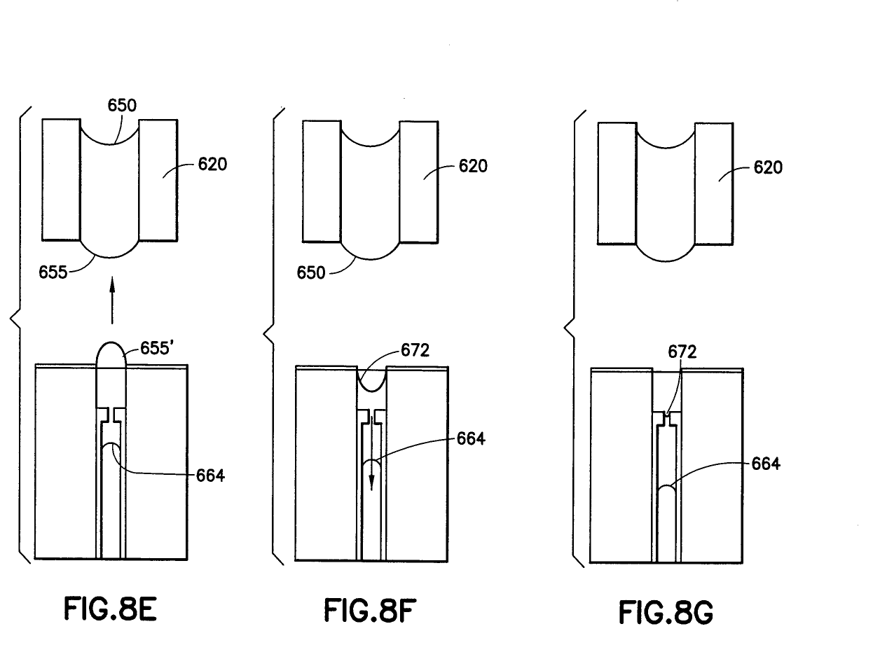

FIG. 10 shows a detail of the interface between lower frame 110 and holding frame 150. On the right side, a pair of actuators 130 at the top and bottom are positioned between lower frame 110 and frame 150. Actuators 130 may be piezoelectric, screws controllable by commands from a controller not shown and pistons activated by compressed gas, etc. They push frame 150 to the left. Conventional springs or an elastomer on the left of the frame supply restoring force if needed. Optionally, e.g. the piezoelectric actuators can be bonded at both ends and will not need a restoring force. The same arrangement is repeated on the bottom. With this approach, the upper frame can be pushed in the x-y plane to a desired position. The contact surfaces against which the actuators push can be in the same plane as plate 50 or can be offset vertically, at the option of the designer.

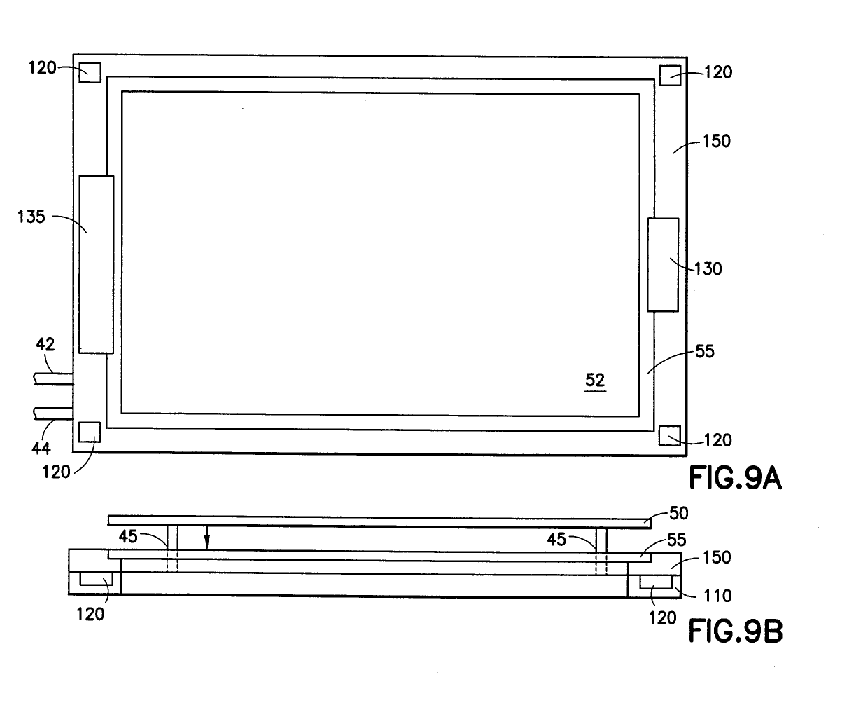

FIG. 11 shows a side view of an alternative embodiment of the invention, in which a second ledge 65 positioned above ledge 55 holds an array of microlenses used for optical examination of the results of the combination of test specimen and reagent in the wells. The lenses can focus light onto the fluids under test and can also deliver light to a commercially available optical device.

The dotted line 75 at the bottom represents an optional lower lens array.

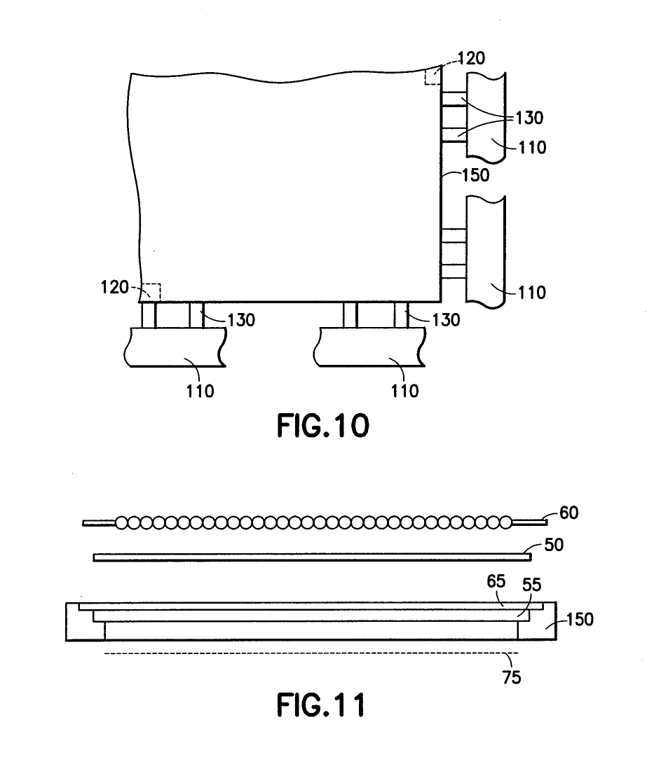

A distribution/operation system can be used to process the microfluidic arrays. In FIG. 12, there is shown in general form an array of units matching the well array and containing a set of rows 72-1-72-n that contain alternating units represented by circles 77 and boxes 78. A set of heavy lines 73-1-73-n represent a distribution system for pressure and/or vacuum. The circular and rectangular symbols 77 and 78, respectively, are used to point out that it is not necessary according to the invention that all units be the same. For example, the boxes could represent a chamber as denoted in FIG. 3 for receiving surplus fluid after a rinsing operation and the boxes could represent a pressure source with individual valve control for applying pressure to the bottom of a module 310 as shown in FIG. 3. As another option, the circles could represent micro-lenses as in FIG. 6, and the boxes represent pressure/vacuum supply.

The plate being processed could have wells that only use one of the two options (or could have a standard array with only half the wells being used for this particular operation). Alternatively, the frame 150 could be translated by the actuators (with the plate optionally being lifted vertically to slide without making contact with the lower array), so that in a first operation, half the wells are processed by circles 77, say, the plate is translated and, in the second operation, the second half of the wells are processed. The two-step process could then be repeated using the devices represented by the rectangles. Alternatively, a first half of the array could be processed with both the circles and rectangles and then the second half.

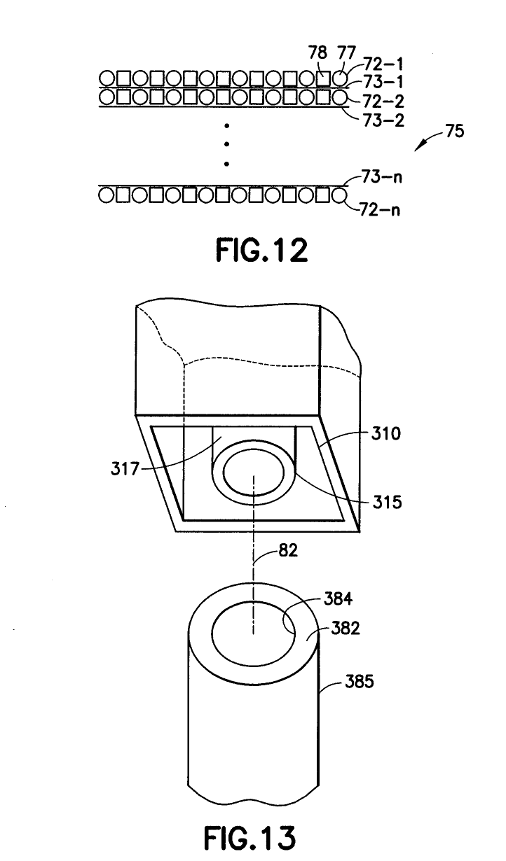

Referring to FIG. 13, there is shown an exploded view of the interface between a module 310, as shown in FIG. 3, and the distribution/operation system. In this version, unit 310 has a projecting cylindrical nozzle 317 having a bottom surface 315 and enclosed by wall 310. Below, the support system represented by dotted line 680 in FIG. 6, has a cylinder 385 with an inner surface 384 and top surface 382. Axis 82 denotes that the two cylinders have a common center. in one embodiment, surface 315 presses against surface 382, with wall 310 projecting past the point where the surfaces meet to confine any spray that may result. In another embodiment, inner surface 384 may enclose the projecting cylinder 317, so that there is vertical overlap. Gas pressure, vacuum or reagents may be supplied from cylinder 385 into the module or may be removed, e.g. a vacuum may be used to draw unused reagent out of the cell, with the result of the reaction either having been determined by optical means or by depositing on the inner wall of cylinder 315, to be tested in a later step. Instead of a cylinder, a wide flat surface as shown in FIG. 6 may be used.

Those skilled in the art will appreciate that the reagent can be urged against the reacting surface (or other reagents in the form of non-homogeneous substances such as microparticles, microbeads, nanoparticles or biological cells) by the application of an external force such as gravity, electrophoretic force or electroosmotic force.

While the invention has been described in terms of a single preferred embodiment, those skilled in the art will recognize that the invention can be practiced in various versions within the spirit and scope of the following claims.

1. A method of reacting a reagent through a plate having a set of vertical apertures for the passage of at least one substance from a first location to a second location, comprising the steps of:
providing a plate having a set of vertical apertures arranged in a array of sample cells;
introducing a first reagent in one of said vertical apertures and reacting said first reagent with a second reagent.

2. A method according to claim 1, in which each sample cell contains at least two vertical apertures connected by a horizontal aperture.

3. A method according to claim 1, in which at least one of said sets of vertical apertures contains removable liners, further comprising removing at least one removable liner and processing material adhering to said removable liners away from said plate.

4. A method according to claim 3, in which at least one of said removable liners is a carrier for a reagent, whereby in operation said reagent reacts with a substance in an applied fluid.

5. A method according to claim 1, in which at least one of said sets of vertical apertures is connected to a space for storing rinsing fluid and further comprising as step of rinsing a substance adhering to a surface of an aperture after a reaction step.

6. A method according to claim 5, in which said space for storing rinsing fluid is connected by differential capillary means that permits passage of said rinsing fluid and blocks passage of said reagent.

7. A method according to claim 1, in which a bottom member is removably attached to said structure, whereby a material adhering to said connecting horizontal aperture may be processed away from said plate.

8. A method according to claim 7, in which at least one of said materials adhering to said set of connecting horizontal apertures is a carrier for a reagent, whereby in operation said reagent reacts with a substance in an applied fluid.

9. A method according to claim 1, in which said vertical apertures and a reaction region of structures of apertures are adapted such that bubbles rise to a region outside said reaction region.

10. A method for reagent delivery to a surface of a microchannel comprising the steps of:
a) bringing the reagent into close proximity with the surface by inserting the reagent in a microstructure whose height is a reduced multiple of the reagent's diffusion constant;
b) reacting the reagent with the desired surface for a reaction time; and
replenishing the microchannel with new reagent when the concentration of reagent near the surface is reduced below a threshold value.

11. A method according to claim 10, where the introduction of new reagent is performed in one of a continuous process and a quantized process.

12. A method according to claim 10, further including a step of depositing lines or spots on a substrate.

13. A method of reacting a reagent through a plate for the passage through a set of apertures of at least one substance from a first location to a second location comprising the steps of:
providing a plate having a set of vertical apertures arranged in a array of sample cells;
introducing a first reagent in one of said vertical apertures and holding a trailing end of said first reagent at a capillary retention valve.

14. A method according to claim 13, further comprising:
introducing a second reagent and reacting said first reagent with said second reagent.

15. A method according to claim 14, further comprising:
introducing said second reagent to said first reagent by means of differential capillary attraction attracting said second reagent toward said first reagent.

16. A sample-holding plate according to claim 14, in which said first and second reagents are positioned such that a measurable portion of one of said first and second reagents is within a diffusion length of the other of said first and second reagents.

17. A sample-holding plate according to claim 15, in which said first and second reagents are positioned such that a measurable portion of one of said first and second reagents is within a diffusion length of the other of said first and second reagents.

18. A method for the delivery of non-homogeneous materials in a fluid to a surface in a microfluidic apparatus comprising the steps of:
delivering a fluid containing non-homogeneous materials to a specified position in a microfluidic apparatus; and
using an external force to enable the interaction of the non-homogeneous materials with a surface wall of the microfluidic apparatus.

19. A method according to claim 18, where the external force is selected from the group comprising gravity, electrophoretic force or electroosmotic force.

20. A method according to claim 18, where the non-homogeneous materials are selected from the group comprising microparticles, microbeads, nanoparticles or biological cells.

21. A method of reacting a reagent through a plate for the passage through a set of apertures of at least one substance from a first location to a second location comprising the steps of:
providing a plate having a set of vertical apertures arranged in a array of sample cells;
introducing a first reagent in one of said vertical apertures and holding a trailing end of said first reagent at a capillary retention valve; and
after a period of time, reacting said first reagent with a second reagent.

22. A method according to claim 21, in which at least one of said sets of vertical apertures is connected to a space for storing rinsing fluid and further comprising a step of rinsing a substance adhering to a surface of an aperture after a reaction step.

23. A method according to claim 21, in which a bottom member is removably attached to said structure, whereby a material adhering to said connecting horizontal aperture may be processed away from said plate.

24. A method of metering the delivery of reagents into a microfluidic component comprising the steps of:
interacting a fluid reservoir with the fluid input of a microfluidic component;
controlling the flow into the device using flow restriction elements in the microfluidic component; and
timing the duration of interaction such that the desired amount of fluid is delivered into the microfluidic component.

25. A method according to claim 24, where the fluid is driven by capillary action.

26. A method according to claim 24, where the fluid in the microfluidic component self-positions itself relative to a capillary retention valve.
